(19) United States
(12) Patent Application Publication
(10) Pub. No.: US 2009/0017463 A1
(43) Pub. Date: Jan. 15, 2009

(54) METHODS FOR PREDICTING PROSTATE CANCER RECURRENCE

(75) Inventor: NEIL A. BHOWMICK, Nashville, TN (US)

Correspondence Address: FULBRIGHT & JAWORSKI L.L.P., 600 CONGRESS AVE., SUITE 2400, AUSTIN, TX, 78701, US

(73) Assignee: VANDERBILT UNIVERSITY

(21) Appl. No.: 12/170,913

(22) Filed: Jul. 10, 2008

Related U.S. Application Data
(60) Provisional application No. 60/948,856, filed on Jul. 10, 2007.

(51) Int. Cl. C12Q 1/68; G01N 33/566

(52) U.S. Cl. 435/6; 435/7.92

(57) ABSTRACT

The present invention relates to the identification of four cytokine biomarkers in prostatic tissue that exhibit differential expression following prostatectomy that, in combination at least one other factor, are able to reliably predict the development of biochemical recurrence following surgery. This marker combination improves the risk stratification of patients after primary local treatment for localized prostate cancer.

The present application claims benefit of priority to U.S. Provisional Application Ser. No. 60/948,856, filed Jul. 10, 2007, the entire contents of which are hereby incorporated by reference.

### BACKGROUND OF THE INVENTION

I. Field of the Invention

The present invention relates to the fields of oncology and pathology. More particular, the invention relates to methods for predicting recurrence of prostate cancer following therapeutic intervention.

II. Related Art

There is a large disparity between the number of newly diagnosed cases of prostate cancer in the United States every year and the number of men who die of the metastatic progression of the disease (Jermal et al., 2004). As a consequence, even though prostate cancer is the second leading cause of cancer related mortality in men in the United States, there is an ongoing concern that the medical community is over-diagnosing, and hence over-treating, the disease. The challenge has been to determine up-front which patients harbor high-risk disease requiring aggressive/curative therapy and which patients harbor indolent disease that could be managed with active surveillance. The issue is an important one given the potential for attempts at local curative therapy (whether it be surgery, radiation or cryotherapy) to subject the patient to both short-term and long-term morbidity.

Currently clinicians rely on a combinatorial assessment of the pre-treatment PSA value, clinical tumor stage, and biopsy Gleason score to risk stratify patients. Yet these methods are unable to distinguish 80% of the patients that may not have any clinical consequences from the prostate cancer (Thompson et al., 2005). In addition to pre-surgical risk stratification, the issue of patient selection for adjuvant therapy after attempted local curative therapy has been taking on increasing importance. In many cases, anti-androgens or other chemotherapeutics are not given to the patients until they develop frank biochemical failure. The risk of progression after local therapy is generally estimated based on available clinicopathologic variables. For example, after radical prostatectomy it is estimated based on both clinical data, such as pre-surgical PSA, as well as pathologic data such as extracapsular extension, seminal vesicle involvement, surgical margin status, and the Gleason score. These approaches described by Amico et al., Kattan et al., and others have improved the inventor's ability to risk stratify patients through a continuous variable taking in to account these types of variables with high specificity, yet relatively low sensitivity (Chun et al., 2006; D'Amico et al., 2000; Kattan, 2003). Due to the lower sensitivity of such available risk stratifying measures it has been difficult to justify the routine use of adjuvant therapy prior to frank biochemical recurrence. Prompt treatment of early metastatic cancers with conventional chemotherapy has shown promise of late (Stephenson and Eastham, 2005; Leibovici et al., 2005; Pienta, 2003). Additionally, several randomized phase III trials have tested the use of routine, adjuvant radiation therapy after prostatectomy in men with high-risk disease estimated using only clinicopathologic parameters (Bolla et al., 2005; Bottke and Wiegel, 2007; Thompson et al., 2006). These studies found significant improvement in biochemical recurrence-free survival in men with adjuvant radiation therapy, but no improvement in overall survival (Morgan et al., 2008). An inability to identify men at high risk may have contributed to the equivocal outcome. This was in no small part due to deficiencies in the inventor's ability to accurately risk-stratify patients using only clinicopathologic variables. However, since adjuvant therapies often provide only temporary suspension of disease progression, the poor efficacy may be because it is administered after development of frank biochemical prostate cancer recurrence (Akduman and Crawford, 2003; Glode, 2006; Gomella et al., 2003). Since up to 35% of men experience PSA recurrence following surgical treatment, indicating potential metastatic spread of the disease, better forms of early detection and risk stratification would support the more appropriately targeted use of adjuvant therapies (Freedland et al., 2005).

Inflammatory chemokines in the tumor microenvironment can regulate the fate of tumor progression (Kakinuma and Hwang, 2006; Tenta et al., 2005). Inflammatory cell recruitment in prostate cancer has emerged as a modulator of metastatic progression (Karin, 2006; Luo et al., 2007).

### SUMMARY OF THE INVENTION

Thus, in accordance with the present invention, there is provided a method of predicting the post-surgical progression of prostate cancer in a subject comprising (a) assessing the level of CCL4 (MIP1β), CX3CL1 (fractalkine) and IL-15 in a tumor sample; (b) assessing at least one additional prognostic factor; and (c) predicting the post-surgical progression of prostate cancer in said subject. The one additional factor may be pre-surgical PSA, stage, prostate capsule invasion, surgical margin status, seminal vesicle involvement, lymph node involvement, IL-1α, IP-10 level and/or Gleason grade. Step (a) may comprise real-time PCR, immunohistochemistry or ELISA. The tumor sample may be subjected to lectin-based protein enrichment. The method may further comprise making a post-operative treatment or monitoring decision, such as to administer anti-androgens or chemotherapeutics or to perform frequent disease surveillance.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. The histologic evaluation of the frozen prostate cores. (FIG. 1A) Each tissue core was cut longitudinally into thirds for duplicate chemokine enrichment and histology analysis, respectively. Subjects B (FIG. 1B) and C (FIG. 1C) developed biochemical recurrence, but subject D (FIG. 1D) was free of recurrence for the five years following prostatectomy.

FIG. 2. Receiver Operating Characteristics (ROC) curves for the prediction models impacted by chemokines (CCL4, CX3CL1, IL-15). Predicted probability of recurrence for each subject was computed from logistic regression models including pre-operative PSA, surgical margin status, seminal vesicle invasion status, pathologic Gleason score, with and without chemokines. Specificity and sensitivity were computed at each possible cutoff on the predicted probability for the two models. The area under the curve (AUC) were compared for the two models.

FIGS. 3A-C. The Kaplan-Meier estimates of the recurrence-free survival based on chemokine expression. The patients were separated into two groups, divided at median tissue level for CCL4 (FIG. 3A), CX3CL1 (FIG. 3B), and IL-15 (FIG. 3C). The two groups were discriminated by the median respective chemokine expression concentration indicated, termed upper half, those below the median lower half. The recurrence-free survival probabilities were estimated by the Kaplan-Meier method and the differences tested using the log-rank test. Each of the dichotomous chemokine expression levels supported statistically significant differences in biochemical recurrence free survival.

FIG. 4. Cox proportional hazard regression. Multivariable Cox proportional hazard regression for biochemical recurrence free survival demonstrated that preoperative PSA, surgical margin, CCL4 and CX3CL1 to are significant predictors of recurrence-free survival. For each predictor variable, the vertical bars illustrate the hazard ratio estimate and the gray horizontal bars represent the respective 95% confidence intervals. The hazard ratios were computed for a change from the lower quartile to upper quartile in continuous variables, namely, Gleason Score: 6-8, Preoperative PSA: 5-9. CCL4: 0.003 to 0.04, CX3CL1: 0.01 to 0.07, IL-15: 0.004 to 0.06. For both surgical margins and seminal vesicle involvement, negative is the reference group.

FIG. 5. Nomogram from the Cox proportional hazard regression model. The Cox proportional hazard regression model was used to create a prediction model for 1-, 3- and 5-year recurrent-free survival. A value in each predictor variable corresponds to a point scale (top). The sum of the individual predictor variable points correspond to the probability of 1-, 3-, and 5-year recurrent-free survival (bottom).

### DETAILED DESCRIPTION OF THE PREFERRED EMBODIMENTS

### I. The Present Invention

Inflammatory chemokines in the tumor microenvironment can regulate the fate of tumor progression (Kakinuma and Hwang, 2006; Tenta et al., 2005). Inflammatory cell recruitment in prostate cancer has emerged as a modulator of metastatic progression (Karin, 2006; Luo et al., 2007). The inventor sought to identify factors that mediate inflammatory cell recruitment since the immune response to cancer can result in both tumor cell ablation as well as provide growth factors to further stimulate tumor progression and motility. The nested case-control study described, identify the differential expression of two chemokine biomarkers in prostatic tissue that support greater detection sensitivity for the biochemical recurrence of prostate cancer alone following prostatectomy. However, incorporating clinicopathologic parameters plus three chemokine biomarkers provided superior prediction of biochemically recurrent disease than clinicopathologic parameters alone. The nomogram developed based on the modeling results illustrate the power of the combined survival prediction. The model has the potential to improve patient risk stratification after primary local treatment for prostate cancer and enable earlier decision making for possible secondary therapeutic options.

Cytokines, chemokines, and growth factors in the tumor microenvironment regulate the fate of tumor progression (Kakinuma and Hwang, 2006; Tenta et al., 2005). However, no such secreted factor has been described thus far as a viable biomarker for patients with recurrent prostate cancer. This is likely due to: (i) the few antibodies available for the immunohistochemical detection of such factors; (ii) cytokines and growth factors can signal under low concentrations and most currently available detection methods are unlikely to identify species of low abundance directly (e.g., mass spectrometry); and (iii) cDNA arrays suggest gene expression may not be a reliable signature of the protein expressed in the local microenvironment. The inventor hypothesized that tissue chemokines can be strong biomarker candidates for distinguishing patients with high risk for biochemical recurrence or metastatic progression of prostate cancer.

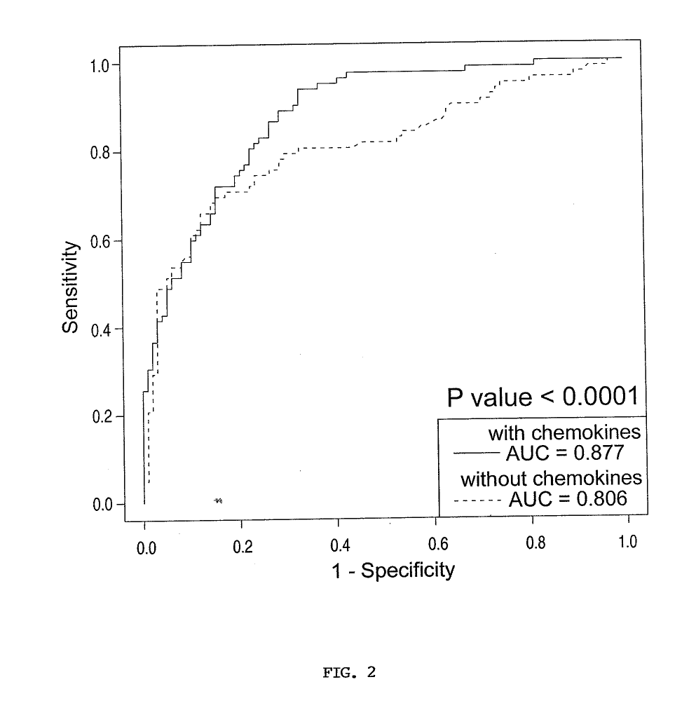

In most cases, patients with clinically localized prostate cancer treated locally with modalities such as surgery or radiation therapy will be cured of their disease. However, a proportion of men will harbor microscopic localized or metastatic residual disease. These patients will ultimately go on to develop biochemical recurrence of disease and eventually are at risk of developing clinical metastatic progression and death from their prostate cancer. The risk of progression after local therapy is generally estimated based on available clinicopathologic variables. For example, after radical prostatectomy it is estimated based on both clinical data, such as pre-surgical PSA, as well as pathologic data such as extracapsular extension, seminal vesicle involvement, surgical margin status, and the Gleason score. These approaches described by D'Amico et al. (2000), Kattan et al. (2003), and others (Chun et al., 2006) have improved the inventor's ability to risk stratify patients through a continuous variable taking in to account these types of variables with high specificity, yet relatively low sensitivity. Due to the lower sensitivity of such available risk stratifying measures it has been difficult to justify the routine use of adjuvant therapy prior to frank biochemical recurrence. Several randomized phase III trials have tested the use of routine, adjuvant radiation therapy after prostatectomy in men with high-risk disease estimated using only clinicopathologic parameters (Bolla et al., 2005; Bottke and Wiegel, 2007; Thompson et al., 2006). These studies found significant improvement in biochemical recurrence-free survival in men with adjuvant radiation therapy, but no improvement in overall survival (Morgan et al., 2008). An inability to identify men at high risk may have contributed to the equivocal outcome. This was in no small part due to deficiencies in the inventor's ability to accurately risk-stratify patients using only clinicopathologic variables. With the addition of the chemokines identified in this study to more standard clinicopathologic variables, the inventor was able to predict the risk of recurrence among men who after prostatectomy with greater accuracy than by clinicopathologic parameters alone. The logistic regression model that included the three chemokines improved the specificity from 36% to 72% at 90% sensitivity, when compared to clinicopathologic parameters alone (FIG. 2). This corresponded with a significant improvement in the AUC for the model that included all three chemokines to 87.7% versus 80.6% for only clinicopathologic variables.

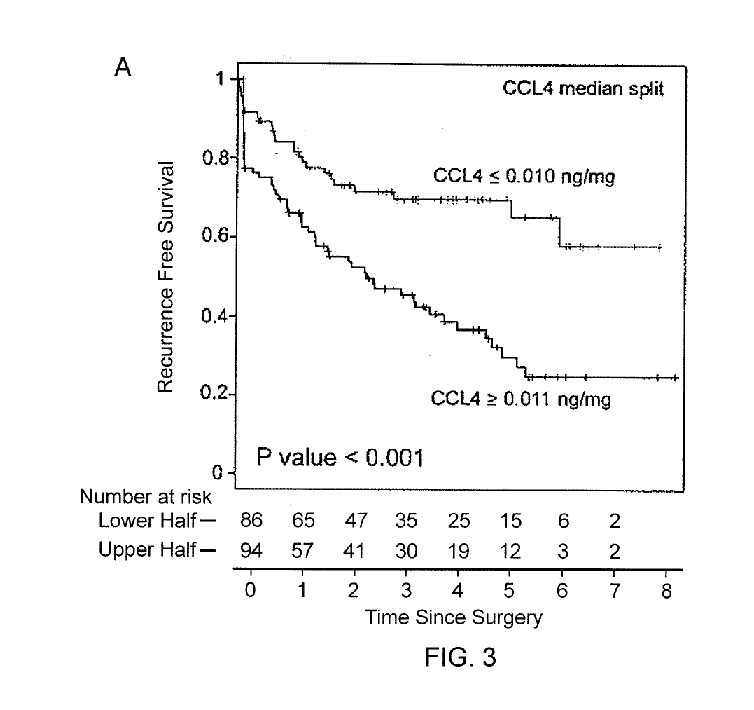

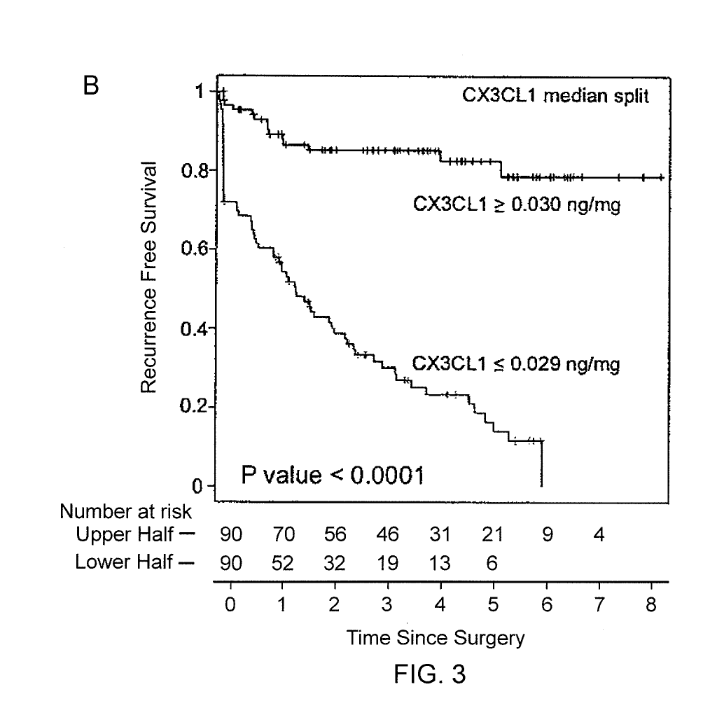

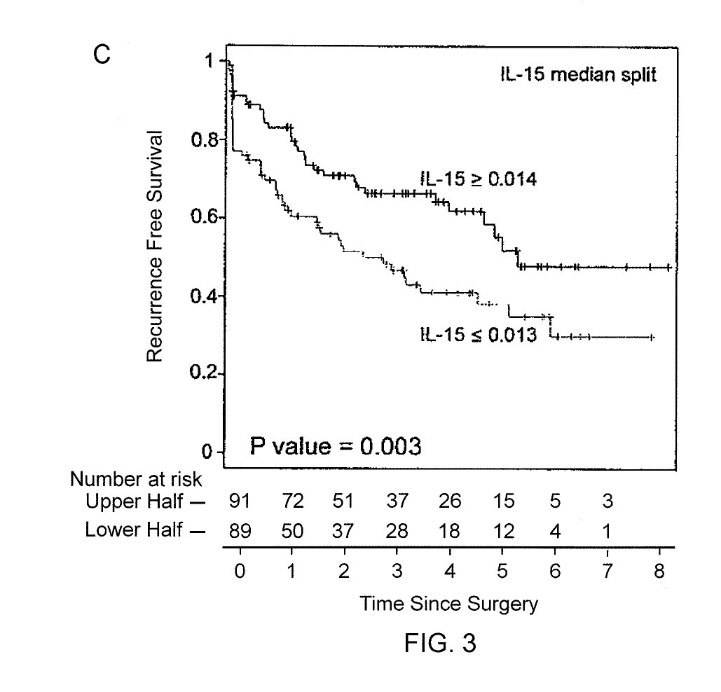

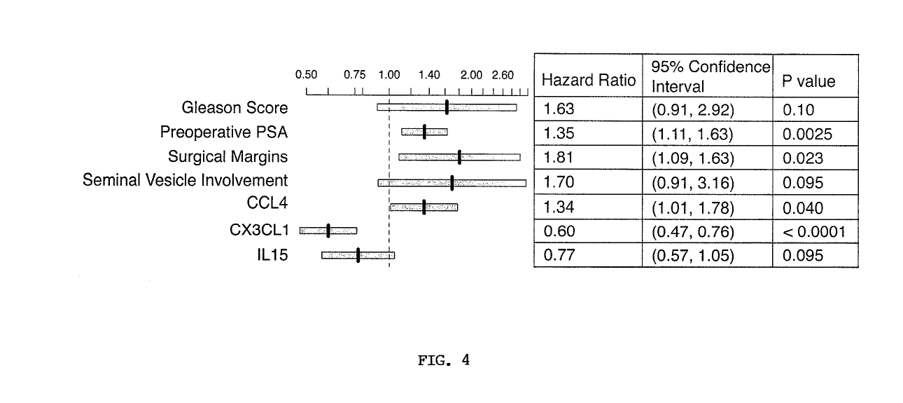

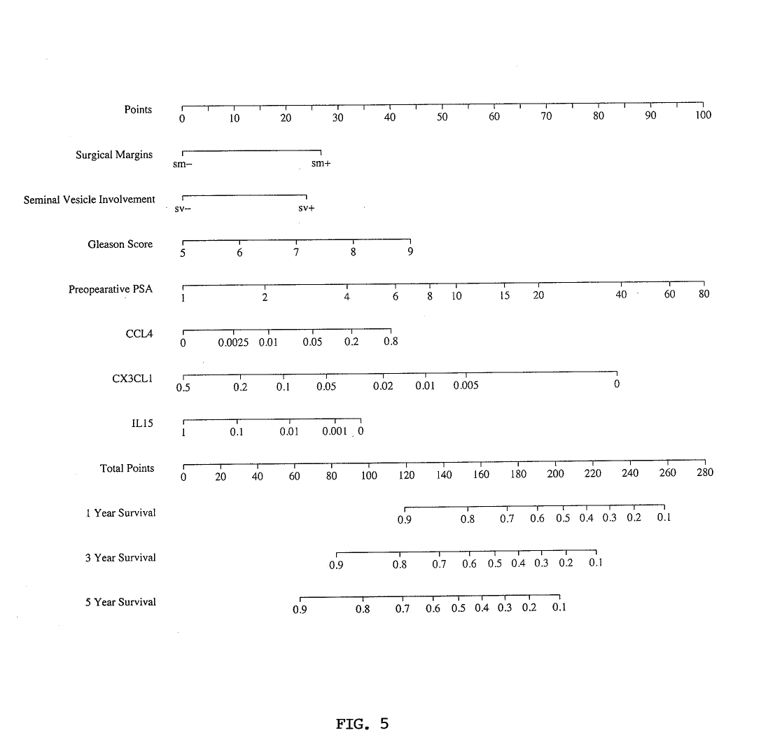

The chemokines identified through the lectin-based enrichment method have specific positive and negative biological roles in prostate cancer progression. All eukaryotic organisms glycosylate proteins exposed to the extracellular space. Since lectins specifically bind such glycosylation groups, the inventor processed the prostatectomy specimens with wheat germ agglutinin resin in batch and were able to observe the expression of a number chemokines otherwise not detectible. Inflammatory cells are emerging as potential mediators of cancer metastasis (Luo et al., 2007). Both CX3CL1 and CCL4 can recruit NK cells, T cells, and monocytes. Based on these data, however, the two chemokines seem to reflect an opposite status of prostate cancer recurrence. Accordingly, apart from similar inflammatory recruitment characteristics, CCL4 has direct proliferative and migration effects on prostate cancer cells in vitro (Akashi et al., 2006). Conversely, CX3CL1 is reported to reduce migration of prostate cancer cells in culture (Shulby et al., 2004). Finally, IL-15 can prevent prostate cancer progression by supporting NK-cell function in vivo (Suzuki et al., 2001; Wu et al., 2004). CX3CL1 and CCL4 overwhelmingly was supported prediction of prostate cancer biochemical recurrence under all univariant and multivariant criteria tested. Although the univariable analysis suggested IL-15 to be significant, as illustrated by Kaplan-Meir plot (FIGS. 3A-C), the overall multivariable models (logistic regression and Cox proportional hazard) did not indicate statistical significance in predicting recurrence-free survival (Table 2, FIG. 4). Nevertheless, specific multivariable one, three, and five-year survival analysis supported the importance of IL-15 as a predictive factor, illustrated in the nomogram (FIG. 5). The significant contribution of each of the chemokine biomarkers needs to be taken in context of clinicopathologic parameters. The well-recognized predictors for biochemical recurrence, like Gleason scores and seminal vesicle involvement, were greater in the population studied (Table 1), however their predictive significance was diminished when compared to the chemokine biomarkers in a multivariable analysis (FIG. 4). Together, the data argue for considering multiple biomarkers in the prediction of those prostate cancers likely to have the greatest clinical significance, especially when coupled to clinicopathologic parameters.

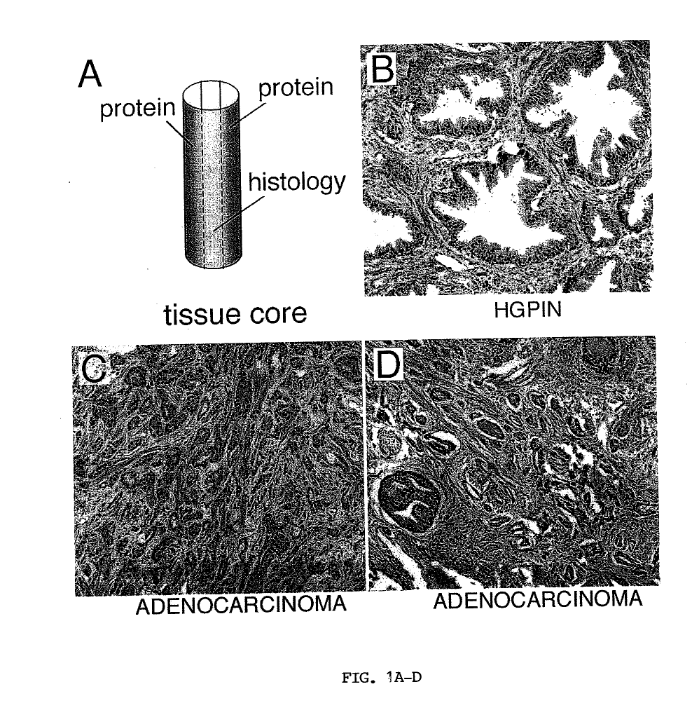

The inventor has shown in this report that a cumulative evaluation of the expression by a continues variable was able to accurately predict biochemical recurrence after radical prostatectomy. Using a model incorporating chemokines and clinicopathologic variables developed in this study, men could be identified who were at substantially higher risk of recurrence after prostatectomy. While local recurrence can potentially be treated with prostatic bed salvage radiation, systemic disease is treated with hormonal therapy, of which the exact timing is controversial. However, of the 82 subjects with biochemical recurrence, 44 (54%) showed elevated PSA within one year of surgery. These apparent early recurrence subjects likely had already developed local or distant metastasis prior to surgery. In an era where there is growing concern that many prostate cancers are over-treated, the use of this model could support the clinical determination of those prostate cancers that would progress to symptomatic disease and ultimately death, as well as potentially provide novel biologic targets to inhibit the metastatic progression. At the same time, it has the potential to identify those patients who harbor more indolent disease that could be managed by active surveillance and may be spared the potential morbidity of aggressive local therapy. In principal, the same model that was generated in this study could be done on diagnostic biopsy specimens and incorporated into a model utilizing clinical variables such as the PSA, clinical Gleason Score, and clinical stage to more accurately predict a patient's disease risk prior to any therapy. It should be noted, however, that this idea was not formally tested in this study as the specimens were obtained on freshly removed prostates after prostatectomy. Nevertheless, the cores utilized here were of similar nature as would be obtained at the time of diagnostic biopsy of the prostate. Notably, many of the tissues processed had no evidence of adenocarcinoma in the sample (FIGS. 1B-D). As the biomarkers are secreted factors, it seems that actual tumor sampling is not particularly necessary. However, more studies are needed to know the extent of adjacency required for positive prediction ability. Therefore, the same techniques used in this study can be readily transferred to the clinical setting at the time of prostate biopsy. Previously reported blood or tissue biomarker analysis have not approached the sensitivity and specificity of the prediction achieved by the model described here (Gonzalez et al., 2004; Henshall et al., 2006; Jayachandran et al., 2008; Paris et al., 2005; Schmidt et al., 2006; Simmons et al., 2007; Ward et al., 2004).

In summary, the inventor has shown that prostate tissue levels of three chemokines: CCL4, CX3CL1, and IL-15, are predictive of biochemical recurrence in men who have undergone radical prostatectomy for adenocarcinoma of the prostate with high specificity and sensitivity. Further, the inventor has shown that a nomogram that incorporates these three chemokines with other established clinicopathologic variables is a better predictor of outcome than using clinicopathologic variables alone. This suggests that CCL4, CX3CL1, and IL-15 are biomarkers of prostate cancer recurrence after radical prostatectomy.

### II. Prostate Cancer

Prostate cancer is a disease in which cancer develops in the prostate, a gland in the male reproductive system. Cancer occurs when cells of the prostate mutate and begin to multiply out of control. These cells may spread (metastasize) from the prostate to other parts of the body, especially the bones and lymph nodes. Prostate cancer may cause pain, difficulty in urinating, erectile dysfunction and other symptoms. Prostate cancer develops most frequently in men over fifty, and is the most common type of cancer in men in the United States, where it is responsible for more male deaths than any other cancer, except lung cancer. However, many men who develop prostate cancer never have symptoms, undergo no therapy, and eventually die of other causes.

Prostate cancer is most often discovered by physical examination or by screening blood tests, such as the PSA (prostate specific antigen) test. Suspected prostate cancer is typically confirmed by removing a piece of the prostate (biopsy) and examining it under a microscope. Further tests, such as X-rays and bone scans, may be performed to determine whether prostate cancer has spread. Prostate cancer can be treated with surgery, radiation therapy, hormonal therapy, occasionally chemotherapy, proton therapy, or some combination of these. The age and underlying health of the man as well as the extent of spread, appearance under the microscope, and response of the cancer to initial treatment are important in determining the outcome of the disease. Since prostate cancer is a disease of older men, many will die of other causes before a slowly advancing prostate cancer can spread or cause symptoms. This makes treatment selection difficult. The decision whether or not to treat localized prostate cancer (a tumor that is contained within the prostate) with curative intent is a patient trade-off between the expected beneficial and harmful effects in terms of patient survival and quality of life.

### III. Targets

A. Protein Marker Set
(i) CCL4

Macrophage Inflammatory Proteins (MIP) belong to the family of chemotactic cytokines known as chemokines. In humans, there are two major forms, MIP-1α and MIP-1β that are now officially named CCL3 and CCL4 respectively. Both are major factors produced by macrophages after they are stimulated with bacterial endotoxins. They activate human granulocytes (neutrophils, eosinophils and basophils) which can lead to acute neutrophilic inflammation. They also induce the synthesis and release of other pro-inflammatory cytokines such as interleukin 1 (IL-1), IL-6 and TNF-α from fibroblasts and macrophages. The gene for CCL4 is both located on human chromosome 17.
(ii) CX3CL1

Chemokine (C-X3-C motif) ligand 1 (CX3CL1) is a small cytokine, which is the only known member of the the CX₃C chemokine family. It is also commonly known under the names fractalkine (in humans) and neurotactin (in mice). The polypeptide structure of CX3CL1 differs from the typical structure of other chemokines. For example, the spacing of the characteristic N-terminal cysteines differs; there are three amino acids separating the initial pair of cysteines in CX3CL1, with none in CC chemokines and only one intervening amino acid in CXC chemokines. CX3CL1 is produced as a long protein (with 373-amino acid in humans) with an extended mucin-like stalk and a chemokine domain on top. The mucin-like stalk permits it to bind to the surface of certain cells. However, a soluble (90 kD) version of this chemokine has also been observed. Soluble CX3CL1 potently chemoattracts T cells and monocytes, while the cell-bound chemokine promotes strong adhesion of leukocytes to activated endothelial cells, where it is primarily expressed. CX3CL1 elicits its adhesive and migratory functions by interacting with the chemokine receptor CX3CR1. Its gene is located on human chromosome 16 along with some CC chemokines known as CCL17 and CCL22.
(iii) IL-15

Interleukin 15 (IL-15) is a cytokine with structural similarity to IL-2 that is secreted by mononuclear phagocytes (and some other cells) following infection by virus(es). This cytokine induces cell proliferation of natural killer cells; cells of the innate immune system whose principal role is to kill virally-infected cells. Maintenance of memory cells does not appear to require persistence of the original antigen; instead, survival signals for memory lymphocytes are provided by cytokines such as IL-15.
(iv) Other markers

The present invention also utilizes several other markers in combination with the foregoing protein markers. For example, pre-surgical prostate specific antigen (PSA) levels may be measured in the biopsy tissue or in another sample. PSA (also known as kallikrein III, seminin, semenogelase, γ-seminoprotein and P-30 antigen) is a protein produced by the cells of the prostate gland. PSA is present in small quantities in the serum of normal men, and is often elevated in the presence of prostate cancer and in other prostate disorders. Higher than normal levels of PSA are associated with both localized and metastatic prostate cancer (CaP). PSA is manufactured almost exclusively by the prostate gland, and is produced for the ejaculate where it liquifies the semen and allows sperm to swim freely. It is also believed to be instrumental in dissolving the cervical mucous cap, allowing the entry of sperm. Biochemically it is a serine protease (EC 3.4.21.77) enzyme, the gene of which is located on the nineteenth chromosome (19q13).

Interleukin-1 (IL-1) is one of the first cytokines ever described. Its initial discovery was as a factor that could induce fever, control lymphocytes, increase the number of bone marrow cells and cause degeneration of bone joints. At this time, IL-1 was known under several other names including endogenous pyrogen, lymphocyte activating factor, haemopoetin-1 and mononuclear cell factor, amongst others. It was around 1984-1985 when scientists confirmed that IL-1 was actually composed of two distinct proteins, now called IL-1α and IL-1β. These belong to a family of cytokines known as the interleukin-1 superfamily.

Both IL-Iα and IL-1β are produced by macrophages, monocytes and dendritic cells. They form an important part of the inflammatory response of the body against infection. These cytokines increase the expression of adhesion factors on endothelial cells to enable transmigration of leukocytes, the cells that fight pathogens, to sites of infection and re-set the hypothalamus thermoregulatory center, leading to an increased body temperature which expresses itself as fever. IL-1 is therefore called an endogenous pyrogen. The increased body temperature helps the body's immune system to fight infection. IL-1 is also important in the regulation of hematopoiesis. For the most part, these two forms of IL-1 bind to the same cellular receptor. This receptor is composed of two related, but non-identical, subunits that transmit intracellular signals via a pathway that is mostly shared with certain other receptors. These include the Toll family of innate immune receptors and the receptor for IL-18.

IP-10 (interferon-γ-inducible 10 kD protein) levels also may be examined as part of the present invention. IP-10 is a CXC chemokine with chemoattractant properties for CD4-positive T cells and inhibits early normal and leukemic hemopoietic progenitor proliferation. IP-10 is produced by a wide variety of cell types ranging from neutrophils and monocytes to hepatocytes, endothelial cells and keratinocytes. The cytokine is reported to be involved in a scala of inflammatory pathologies such as HIV encephalitis, cutaneous T cell lymphoma, chronic hepatitis and acute anterior uveitis. Various observations strongly suggest a role for the CXC chemokines IL-8 and IP-10 in the regulation of angiogenic activity in cancer and in idiopathic pulmonary fibrosis.

Gleason grade is another relevant factor to be used in combination. Gleason grade is determined by assessing the tissue sample for (a) how the cells look (on a scale of 1 to 5), and (b) how the cells are arranged (on a scale of 1 to 5). These two numbers are then combined to give a Gleason Grade score of 2-10. A lower Gleason indicates a well-differentiated with a lower potential to spread (2-4). A high Gleason grade indicates a poorly differentiated cancer, with more likely to spread (8-10).

Surgical margin status and seminal vesicle involvement are pathologic factors determined at the time of surgery. The prostate is found within a membrane capsule. The organ in its entirely is removed in a prostatectomy. However, cancer cells may approach the surgical margins of the tissue. Any evidence of this pathologically is considered positive surgical margins. The seminal vesicles, found subadjacent to the prostate are also removed in surgery. Any evidence of prostate cancer in the seminal vesicle is recorded in the pathologic report.

Also contemplated are determining the stage of prostate cancer, prostate capsule invasion, and lymph node involvement.

B. Purification of Proteins

It may be desirable to purify various proteins in accordance with the present invention. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure polypeptide are ion-exchange chromatography, exclusion chromatography; polyacrylamide gel electrophoresis; isoelectric focusing. A particularly efficient method of purifying polypeptides is fast protein liquid chromatography or even HPLC.

Certain aspects of the present invention concern the purification, and in particular embodiments, the substantial purification, of an encoded protein or peptide. The term “purified protein” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein composition that has been subjected to fractionation to remove various other components, and which composition substantially retains its expressed biological activity. Where the term “substantially purified” is used, this designation will refer to a composition in which the protein forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fuctose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

C. Nucleic Acids

Nucleic acids according to the present invention may encode a portion or all of one of the markers discussed above. The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

Naturally, the present invention also encompasses DNA segments that are complementary, or essentially complementary, to the target sequences. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the target nucleic acid segments under relatively stringent conditions such as those described herein.

Sequences of 17-bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or longer are contemplated as well. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

### IV. Diagnostic Procedures

The present invention provide for methods of predicting progression of prostate cancer. More specifically, by assessing the expression of CCL4 CX3CL1 and IL-15 in cancer tissues, one can assess prostate cancer development, progression and/or metastasis. The diagnosis may be made using genetic- (nucleic acid) or immunologic- (protein) based assays, both discussed in greater detail below.

A. Genetic Diagnosis

One embodiment of the instant invention comprises a method for expression of CCL4, CX3CL1 and IL-15 in tumor tissues. Nucleic acid used is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994). Following detection and quantification, one may compare the results seen in a given patient with a statistically significant reference group of patients with prostate cancer progression or lack thereof.

A variety of different assays are contemplated in this regard, including but not limited to, fluorescent in situ hybridization (FISH), direct DNA sequencing, PFGE analysis, Southern or Northern blotting, single-stranded conformation analysis (SSCA), RNAse protection assay, allele-specific oligonucleotide (ASO), dot blot analysis, denaturing gradient gel electrophoresis, RFLP and PCRTM-SSCP.
(i) Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process. In particular embodiments, the probes or primers are labeled with radioactive species (³²P, ¹⁴C, ³⁵S, ³H, or other label), with a fluorophore (rhodamine, fluorescein) or a chemillumiscent (luciferase).
(ii) Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al. (1990) each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[α-thio]-triphosphates in one strand of a restriction site may also be useful in the amplification of nucleic acids in the present invention, Walker et al. (1992).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification method is described in GB Application No. 2 202 328, and in PCT Application No. PCT/US89/01025, each of which is incorporated herein by reference in its entirety, may be used in accordance with the present invention. In the former application, “modified” primers are used in a PCR™-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double-stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single-stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR™” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., (1989), incorporated herein by reference in its entirety.
(iii) Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.
(iv) Separation Methods

It normally is desirable, at one stage or another, to separate the nucleic acids. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).
(v) Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al. (1989). For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences throughout the Killin gene that may then be analyzed by direct sequencing.
(vi) Design and Theoretical Considerations for Relative Quantitative RT-PCR™

Reverse transcription (RT) of RNA to cDNA followed by relative quantitative PCR™ (RT-PCR™) can be used to determine the relative concentrations of specific mRNA species isolated from patients. By determining that the concentration of a specific mRNA species varies, it is shown that the gene encoding the specific mRNA species is differentially expressed.

In PCR™, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ amplification is directly proportional to the starting concentration of the target before the reaction began. By determining the concentration of the amplified products of the target DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR™ products and the relative mRNA abundances is only true in the linear range of the PCR™ reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR™ for a collection of RNA populations is that the concentrations of the amplified PCR™ products must be sampled when the PCR™ reactions are in the linear portion of their curves.

The second condition that must be met for an RT-PCR™ experiment to successfully determine the relative abundances of a particular mRNA species is that relative concentrations of the amplifiable cDNAs must be normalized to some independent standard. The goal of an RT-PCR™ experiment is to determine the abundance of a particular mRNA species relative to the average abundance of all mRNA species in the sample. In the experiments described below, mRNAs for β-actin, asparagine synthetase and lipocortin II were used as external and internal standards to which the relative abundance of other mRNAs are compared.

Most protocols for competitive PCR™ utilize internal PCR™ standards that are approximately as abundant as the target. These strategies are effective if the products of the PCR™ amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR™ assay for clinically derived materials. The problems inherent in clinical samples are that they are of variable quantity (making normalization problematic), and that they are of variable quality (necessitating the co-amplification of a reliable internal control, preferably of larger size than the target). Both of these problems are overcome if the RT-PCR™ is performed as a relative quantitative RT-PCR™ with an internal standard in which the internal standard is an amplifiable cDNA fragment that is larger than the target cDNA fragment and in which the abundance of the mRNA encoding the internal standard is roughly 5-100-fold higher than the mRNA encoding the target. This assay measures relative abundance, not absolute abundance of the respective mRNA species.

Other studies may be performed using a more conventional relative quantitative RT-PCR™ assay with an external standard protocol. These assays sample the PCR™ products in the linear portion of their amplification curves. The number of PCR™ cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR™ assays can be superior to those derived from the relative quantitative RT-PCR™ assay with an internal standard.

One reason for this advantage is that without the internal standard/competitor, all of the reagents can be converted into a single PCR™ product in the linear range of the amplification curve, thus increasing the sensitivity of the assay. Another reason is that with only one PCR™ product, display of the product on an electrophoretic gel or another display method becomes less complex, has less background and is easier to interpret.
(vii) Chip Technologies

Specifically contemplated by the present inventor are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al. (1994); Fodor et al. (1991).

B. Immunodiagnosis

Antibodies of the present invention can be used in characterizing the content of CCL4, CX3CL1 and IL-15 in cancer tissues through techniques such as ELISAs and Western blotting. The use of antibodies to CCL4, CX3CL1 and IL-15 in an ELISA assay is contemplated. For example, antibodies may be immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for the target that differs the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2-4 hrs, at temperatures preferably on the order of about 25°-27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present invention will also find use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel- or fluorescently-tagged secondary antibodies against the toxin moiety are considered to be of particular use in this regard.

### V. Kits

According to the present invention, there are provided kits for assessing the expression of CCL4, CX3CL1 and IL-15. The kit of the present invention can be prepared by known materials and techniques which are conventionally used in the art. Generally, kits comprises separate vials or containers for the various reagents, such as probes, primers, antibodies, etc. The reagents are also generally prepared in a form suitable for preservation by dissolving it in a suitable solvent. Examples of a suitable solvent include water, ethanol, various buffer solutions, and the like. The various vials or containers are often held in blow-molded or injection-molded plastics.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Patient selection. This study was conducted in accordance with the Institutional Review Board, Vanderbilt University. The digital medical record of 660 subjects was retrospectively examined using the Vanderbilt University Urologic Surgery registry of radical prostatectomies performed between 1998 and 2002. Several of these patients were excluded for reasons that included, availability of at least five-year follow-up data, availability of archived fresh frozen peripheral zone tissue, and records of pre-surgical hormone ablation therapy. Patients who had undergone hormone ablation therapy at any point prior to surgery or the demonstration of biochemical recurrence were excluded. Biochemical recurrence following prostatectomy was defined as PSA≧0.2 ng/ml confirmed at least once with another PSA at least two weeks apart, and associated with two consecutive subsequent increases in PSA level. Ultimately, for this nested study the inventor focused on 82 subjects who developed biochemical recurrence within five years of prostatectomy and an age matched control group of 98 subjects who were free of recurrence within the same time frame. The mean age for the subjects was 60 years (range 43-72 years). All subjects were annotated based on age, race, pre-surgical serum PSA, pathologic Gleason score, pathologic stage, extracapsular involvement, seminal vesicle involvement, surgical margin status, and detection of biochemical recurrence (Table 1).

Sample preparation and analysis. The tissue samples were derived from a tumor bank of frozen cores of the prostate from patients after radical prostatectomy for adenocarcinoma of the prostate. Eight 4 mm diameter cores were taken from fresh prostates removed during prostatectomy and snap frozen in liquid nitrogen. Four of these cores were from the peripheral zone, where the majority of the prostate cancer originates. The frozen cores, determined by gross assessment of tumor involvement, were dissected longitudinally into three sections with the outside sections used for duplicate protein isolation (FIG. 1A). The central piece was paraformaldehyde fixed and paraffin embedded for histologic evaluation by a pathologist.

Samples were homogenized in lysis buffer (100 mM Tris pH 7.2, 500 mM NaCl), sonicated and centrifuged for 5 minutes at 1000×g. Glycosylated proteins were purified using the glycoprotein isolation kit, WGA (Pierce Biotechnology, Inc.) according to the manufacturer's instructions. All procedures except for binding to the wheat germ agglutinin resin (Pierce Biotechnology, Inc.) were performed on ice in siliconized microcentrifuge tubes. The expression levels of thirty chemokines of the resulting samples (100 μl) were measured by LINCO Research Inc. (Billerica, Mass.) using the human chemokine multiplex antibody-array for: TGF-β1, IL-1α, IL-1β, IL1α receptor, IL-2, IL-4, IL-5, IL-6, IL-7, IL-8, IL-10, IL-12p40, IL-12p70, IL-13, IL-15, IL-17, EGF, TGF-α, CX3CL1, CCL2, sCD40L, IP-10, VEGF, RANTES, GM-CSF, G-CSF, IFN-γ, MIP1α, MIP1β, Eotaxin. The expression levels were normalized to total protein.

Statistical Analysis. Baseline patient characteristics. Baseline demographic and clinical variables for all patients were assessed using Wilcoxon rank sum tests for continuous variables and Fisher exact tests for categorical variables. Biomarker expression levels underwent logarithmic transformation to stabilize variance. Values below the detection limit were imputed with half of the minimum detected value for the particular biomarker.

Candidate chemokine selection. As there were 31 candidate chemokines, it was necessary to reduce the number of candidate variables before regression models could be considered. Exploratory data analyses were conducted on 36 (21 recurrent and 15 recurrent-free) prostatectomy subjects. The strength of marginal relationship to the response was used to eliminate variables that showed only a very weak relation. Spearman's rank correlations and Wilcoxon rank sum tests were used for initial screening. A Receiver Operating Characteristics (ROC) curve was constructed for each chemokine and the area under the ROC curve (AUC) was used to compare how strongly chemokines were related to the recurrence. Finally, a backward elimination model building strategy on 1,000 bootstrapped data was utilized to choose variables for further consideration. Using this bootstrap sample, a full model with all the variables in consideration was constructed. Then, the variable with the largest P value (Wald test) was dropped, and a new model was fitted with one fewer variable. Variable elimination continued until all the remaining variables showed a P value smaller than 0.5. The number retentions of each variable in the final model's 1,000 iterations were used to select the variables for further consideration. Pair-wise Spearman's rank correlations among the candidates were also considered in selecting these variables. All the information from these multiple analyses was used to select the candidate chemokines for the next phase of the data collection and model building.

Logistic regression. Demographic and clinical variables for the logistic regression model were selected based on their relation to the outcome variable (biochemical recurrence) and inter-relations among the covariates. Two models were constructed: 1) The three selected chemokine variables with the clinicopathologic variables made up one model. 2) The inventor also fitted a clinical variable only simpler model without any chemokine variables. These two models were compared using a likelihood ratio test. From each model, the predicted probabilities of recurrence were computed and the ROC curves were constructed. Comparison of the ROC curves was conducted with the integrated discrimination improvement (IDI) (Pencina et al., 2008). An estimate of the odds ratio with a confidence interval was reported for each variable based on the developed model.

Survival analysis. To visualize the association between biochemical recurrence-free survival and each chemokine marker, the product limit estimator was computed for the two groups defined as above and below the median, and the logrank tests were used to assess the difference of the recurrence-free survival between the two groups illustrated by Kaplan-Meier plots. A Cox proportional hazard regression was used to model the recurrence-free survival. The proportionality of the hazard ratio was assessed graphically and numerically using Schoenfeld's partial residuals (Schoenfeld, 1983). The effects of predictors in the model were presented with individual hazard ratios. A nomogram equating each of the predictors to the probabilities of 1-year, 3-year, and 5-year recurrence-free survival is also presented. All analyses were carried out with R version 2.7.0 (R RDCT, 2008).

TABLE 1
Clinical and pathologic stratification of the biochemical recurrent and recurrent-
free subject groups (n = 180)

| | Overall | Recurrent-Free | Recurrent | |
|---|---|---|---|---|
| | (n = 180) | (n = 98) | (n = 82) | P value |
| Age | 60.0 (56.0, 66.0) | 60.0 (55.3, 66.0) | 60.5 (56.0, 66.) | P = 0.834¹ |
| Race | | | | |
| -white | 92% (165) | 88% (86) | 96% (79) | P = 0.093² |
| -black | 7% (13) | 10% (10) | 4% (3) | |
| -others | 1% (2) | 2% (2) | 0% (0) | |
| Extracapsular inv. | 42% (76) | 29% (28) | 59% (48) | P < 0.001² |
| Positive margin | 28% (50) | 11% (11) | 48% (39) | P < 0.001² |
| Seminal ves. inv. | 16% (28) | 2% (2) | 32% (26) | P < 0.001² |
| Lymph node inv. | 5% (9) | 0% (0) | 11% (9) | P < 0.001² |
| Preoperative PSA | 6.3% (4.8, 9.1) | 5.7% (4.6, 7.4) | 7.3% (5.1, 13.4) | P < 0.001² |
| Gleason | | | | |
| -5-6 | 24% (44) | 34% (33) | 13% (11) | P < 0.001² |
| -7 | 58% (105) | 59% (58) | 57% (47) | |
| -8-9 | 18% (31) | 7% (7) | 30% (24) | |
| Clinical Stage | | | | |
| -T1c | 63% (114) | 68% (67) | 57% (47) | P = 0.092 |
| -T2a | 25% (45) | 24% (24) | 26% (21) | |
| -T2b+ | 12% (21) | 7% (7) | 17% (14) | |

For continuous variables, a, b, and c represent the median a, lower quartile b, and the upper quartile c.
Numbers after percents are frequencies in parenthesis.
Tests used: ¹Wilcoxon test; ²Fisher's test.

### Example 2

### Results

Chemokines support prediction of biochemical recurrence. The histology of prostatic tissue cores from prostatectomy subjects had focal adenocarcinoma and HGPIN as illustrated in FIGS. 1A-D. However, the histologic patterns were not predictive of future progression to biochemical recurrence. There was no statistical difference in the age, race, and clinical stage of the subjects in the recurrent and recurrent-free groups (Table 1). Since chemokines and growth factors that influence metastatic progression are commonly in low abundance, the inventor developed a methodology to enrich such factors from tissue lysates. Following lectin enrichment of 0.03-0.05 g (wet weight) tissue, 31 chemokines were screened by multiplex ELISA for a panel of inflammatory chemokines. Importantly, little to no signal was detected for these markers if wheat germ agglutinin-mediated enrichment process was not performed on the tissue extracts. The use of another lectin, concanavalin A-mediated enrichment was ineffective in chemokine detection (data not shown). The results of the initial screening of 36 prostatectomy subjects (21 recurrent and 15 recurrent-free) were analyzed by Spearman's rank correlations and Wilcoxon rank sum tests. The common inflammatory factors found to be differentially expressed by both tests in biochemically recurrent and recurrent-free subjects included: CX3CL1, IL-12, IL-15, IL-4, and CCL4. Of this group CCL4 (macrophage inflammatory protein-1β) was up regulated in patients that developed biochemical recurrence within five years following prostatectomy. However, CX3CL1 (fractalkine), IL-12, IL-15, and IL-4 were predominantly down regulated in such high-risk patients. Interestingly, each of the factors has been implicated biologically to the progression of prostate cancer in the past (Jamieson et al., 2008; Lee et al., 2008; Takeshi et al., 2005; Wang and Thompson, 2008; Wu et al., 2004). The area under the ROC curve for individual chemokines and backward elimination bootstrap methods were correlated to biochemical recurrence in finally selecting CX3CL1, CCL4, and IL-15 for further analyses (data not shown). CX3CL1 was the best predictor of recurrence with all the selection methods, and the other two were among the favorable ones with multiple methods.

Validation of chemokine biomarkers in model development. The inventor sought to develop a multivariable logistic regression model for the prediction of the probability of biochemical recurrence following prostatectomy. The inventor considered all predictor variables available to us that were either numerical or categories for logistic regression analysis. In addition to the three chemokine markers, the candidate clinical and surgical variables the inventor considered were pathologic Gleason score, pre-operative PSA, surgical margin status, seminal vesicle involvement, clinical stage, extracapsular involvement, and lymph node metastasis. However, for this population the clinical stage categorized to T1c, T2a, and T2b or greater did not provide any discrimination regarding the recurrence status of the subjects. Extracapsular involvement was also dropped as a covariate as it was highly correlated with surgical margin (Spearman's rank correlation=0.47); there were only 46 cases (25%) for which these two variables do not agree. The limited number of subjects having positive lymph node tumor involvement (n=9) provided little information and was also not considered in further analysis. A fitted logistic regression model of the inventor's population suggested two chemokines, CCL4 (P=0.011) and CX3CL1 (P<0.0001), along with surgical margin (P=0.008), seminal vesicle involvement (P=0.016), pre-operative PSA (P=0.041), and Gleason score (P=0.048) to be significant factors, while IL-15 (P=0.66) was not. Table 2 summarizes the fitted logistic regression model in terms of the odds ratios.

TABLE 2
Odds ratio and confidence interval for the logistic regression model

| | | | Odds | 95% Confidence | |
|---|---|---|---|---|---|
| | Reference | | Ratio | Interval | P Value |
| Gleason score | 6 | 8 | 2.60 | (1.01, 6.71) | 0.048 |
| Pre-operative | 5 | 9 | 1.54 | (1.02, 2.34) | 0.041 |
| PSA | | | | | |
| Surgical margin | negative | positive | 3.64 | (1.41, 9.42) | 0.001 |
| Seminal vesicle | negative | positive | 7.15 | (1.44, 35.5) | 0.016 |
| inv. | | | | | |
| CCL4 | 0.003 | 0.04 | 2.16 | (1.19, 3.91) | 0.011 |
| CX3CL1 | 0.01 | 0.07 | 0.35 | (0.21, 0.58) | <0.0001 |
| IL-15 | 0.004 | 0.06 | 0.88 | (0.51, 1.55) | 0.66 |

Odds ratios for continuous variable represents change from the lower quartile to upper quartile except for Gleason score whose lower and upper quartiles are both 7.

To evaluate the logistic regression model that used only the clinical variables, namely, Gleason Score, pre-operative PSA, seminal vesicle involvement and surgical margin status was compared to the model combining clinical and chemokine variables. The improvement by the three biomarkers was highly significant (likelihood ratio test P value<0.0001). FIG. 2 shows the ROC curves obtained by the predicted values using the two models. Improvement in area under the curve (AUC) was 7.1 percentage points (from 80.6% to 87.7%). Integrated Discrimination Improvement (IDI) was estimated to be 0.116 (P<0.0001) supporting the statistical significance of the improvement (Pencina et al., 2008). The addition of the chemokine biomarkers to the clinical variables provided little improvement in predictive ability up to about 80% sensitivity. However, given a sensitivity of 90%, the clinical variables alone provided a specificity of only 36% (95% confidence interval: 20%, 58%) compared to the addition of the chemokines provided a specificity of 72% (95% confidence interval: 55%, 84%). The addition of the chemokine markers to the clinical variables doubled the specificity at 90% sensitivity (P=0.02) according to ROC analysis. The chemokines, particularly CCL4 and CX3CL1, supported the dichotomous prediction of biochemical recurrence and recurrent-free survival following prostatectomy.

Analysis of Recurrence-free Survival. To define the efficacy of the markers in predicting recurrent-free survival, the same clinical and chemokine variables as in the logistic regression were considered for a Cox proportional hazard regression analysis. CCL4, CX3CL1, and IL-15 proved to individually serve as highly significant markers for biochemical recurrence status by the Kaplan-Meier method (FIGS. 3A-C). Using a dichotomous median split for the upper and lower concentration range for tissue chemokine expression, CX3CL1 exhibited the best prediction ability (P<0.0001) followed by CCL4 (P<0.001) and IL-15 (P=0.003). The proportional hazard assumption was tested with scaled Schoenfeld residuals (Grambsch & Therneau, 1994). There was no evidence of violation, as the chi-square tests for trend were not significant for any of the seven variables (surgical margin status, seminal vesicle involvement, Gleason Score, pre-operative PSA, CCL4, and CX3CL1, and IL-15; P values ranging 0.46 to 0.90). The effect of the covariates in the multivariable Cox regression model on recurrence-free survival was summarized with the hazard ratios with 95% confidence intervals in FIG. 4. CCL4 (P=0.040), CX3CL1 (P<0.0001), pre-operative PSA (P=0.0025), and surgical margin (P=0.023) were significant factors. However, Gleason Score (P=0.10), seminal vesicle involvement (P=0.095) and IL-15 (P=0.095) were not significant in the overall multivariate model.

The inventor computed a nomogram from the Cox proportional hazard regression model that connects each predictor and the probabilities of one, three, and five-year recurrence-free survival (FIG. 5). The contributions of CX3CL1 and preoperative PSA were further illustrated to be important in predicting recurrence-free survival. The inventor also noted that IL-15, although not statistically significant on multivariable analysis, contributed to the prediction of recurrence-free survival comparable to CCL4, seminal vesicle involvement, and surgical margins within the nomogram. Together, the survival model suggested the use of surgical margin status, seminal vesicle involvement, Gleason Score, pre-operative PSA, CCL4, and CX3CL1, and IL-15 for predicting biochemical recurrence following prostatectomy.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. REFERENCES

The following references, to the extent that they provide exemplary procedural or other details supplementary to those set forth herein, are specifically incorporated herein by reference:
U.S. Pat. No. 4,683,195
U.S. Pat. No. 4,683,202
U.S. Pat. No. 4,800,159
U.S. Pat. No. 4,883,750
U.S. Pat. No. 5,279,721
Akashi et al., Oncol Rep., 16:831-836, 2006.
Akduman and Crawford, J. Urol., 169:1993-1998, 2003.
Bellus, J. Macromol. Sci. Pure Appl. Chem., A31(1): 1355-1376, 1994.
Bolla et al., Lancet, 366:572-578, 2005.
Bottke and Wiegel, Urologia Internatl., 78:193-197, 2007.
Capaldi et al., Biochem. Biophys. Res. Comm., 74(2):425-433, 1977.
Chun et al., Eur. Urol., 49:820-826, 2006.
D'Amico et al., Urology, 55:572-577, 2000.
EPO Appln. 320 308
EPO Appln. 329 822
Fodor et al., Biochemistry, 30(33):8102-8108, 1991.
Freedland et al., Jama, 294:433-439, 2005.
Freifelder, In: Physical Biochemistry Applications to Biochemistry and Molecular Biology, 2nd Ed. Wm. Freeman and Co., NY, 1982.
Frohman, In: PCR Protocols: A Guide To Methods And Applications, Academic Press, N.Y., 1990.
GB Appln. 2 202 328
Glode, J. Urol., 176:S30-S31, 2006.
Gomella et al., Urology, 62(1):46-54, 2003.
Gonzalez et al., Urology, 64:723-728, 2004.
Grambsch and Therneau, Biometrika, 81:515-526, 1994.
Hacia et al., Nature Genet., 14:441-449, 1996.
Henshall et al., J. Natl. Cancer Inst., 98:1420-1424, 2006.
Henshall et al., J. Natl. Cancer Institute, 98:1420-1424, 2006.
Innis, et al., In: PCR Protocols. A guide to Methods and Application, Academic Press, Inc. San Diego, 1990.
Jamieson et al., Cancer res., 68:1715-1722, 2008.
Jayachandran et al., J. Urol., 179:1791-1796, 2008.
Jemal et al., Cancer. J. Clin., 54:8-29, 2004.
Kakinuma and Hwang, J. Leukoc. Biol., 79:639-651, 2006.
Karin, Nature, 441:431-436, 2006.
Kattan, Curr. Opin. Urol., 13:111- 116, 2003.
Kwoh et al., Proc. Natl. Acad. Sci. USA, 86:1173, 1989.
Lee et al., Prostate, 68:85-91, 2008.
Luo et al., Nature, 446:690-694, 2007.
Morgan et al., Radiother. Oncol., 88(1):1-9, 2008.
Ohara et al., Proc. Natl. Acad. Sci. USA, 86:5673-5677, 1989.
Paris et al., Int. J. Biolog. Markers, 20:141-145, 2005.
PCT Appln. WO 88/10315
PCT Appln. WO 89/06700
PCT Appln. WO 90/07641
PCT Appln. PCT/US87/00880
PCT Appln. PCT/US89/01025
Pease et al., Proc. Natl. Acad. Sci. USA, 91:5022-5026, 1994.
Pencina et al., Stats in Meds., 27:157-172, 2008.
Pignon et al., Hum. Mutat., 3: 126-132, 1994.
Sambrook et al., In: Molecular cloning: a laboratory manual, 2^{nd} Ed., Cold Spring Harbor Laboratory Press, Cold Spring Harbor, N.Y., 1989.
Schmidt et al., Cancer Res., 66:8959-8965, 2006.
Schoenfeld, Biometrics, 39:499-503, 1983.
Shoemaker et al., Nature Genetics, 14:450-456, 1996.
Shulby et al., Cancer Res., 64:4693-4698, 2004.
Simmons et al., Europ. Urol., 51:1175-1184, 2007.
Suzuki et al., J. Leukoc. Biol., 69:531-537, 2001.
Takeshi et al., Anticancer Res., 25:4595-4598, 2005.
Tenta et al., J. Musculoskelet. Neuronal. Interact., 5:135-144, 2005.
Thompson et al., J. Urol., 174:553-556, 2005.
Thompson et al., Jama, 296:2329-2335, 2006.
Walker et al., Nucleic Acids Res. 20(7): 1691-1696, 1992.
Wang and Thompson, Gene Therapy, 15:787-796, 2008.
Ward et al., J. Urol., 172:2244-2248, 2004.
Wu et al., J. Clin. Invest., 114:560-568, 2004.
Wu et al., Proc. Natl. Acad. Sci. USA, 86(8):2757-2760, 1989.

What is claimed is:

1. A method of predicting the post-surgical progression of prostate cancer in a subject comprising:
(a) assessing the level of MIP1β, fractalkine and IL-15 in a tumor sample;
(b) assessing at least one additional prognostic factor; and
(c) predicting the post-surgical progression of prostate cancer in said subject.

2. The method of claim 1, wherein said at least one additional factor is pre-surgical PSA, IL-1α expression level, stage, prostate capsule invasion, surgical margin status, seminal vesicle involvement, lymph node involvement, IP-10 level and/or Gleason grade.

3. The method of claim 1, wherein said at least one additional factor is pre-surgical PSA

4. The method of claim 1, wherein said at least one additional factor is Gleason grade

5. The method of claim 1, wherein said at least one additional factor is IP-10 level.

6. The method of claim 1, wherein step (a) comprises real-time PCR.

7. The method of claim 1, wherein step (a) comprises immunohistochemistry.

8. The method of claim 1, wherein step (a) comprises ELISA.

9. The method of claim 8, wherein said tumor sample is subjected to lectin-based protein enrichment.

10. The method of claim 1, further comprising making a post-operative treatment or monitoring decision.

11. The method of claim 10, wherein said treatment decision is to administer anti-androgens or chemotherapeutics.

12. The method of 10, wherein said monitoring decision is to perform frequent disease surveillance.
